(19) United States
(12) Patent Application Publication
(10) Pub. No.: US 2006/0211948 A1
(43) Pub. Date: Sep. 21, 2006

(54) Dynamic technique for fitting heart pacers to individuals

(75) Inventors: Menachem Levanoni, Poway, CA (US); Jerome M. Kurtzberg, Yorktown Heights, NY (US)

Correspondence Address: Stephen C. Kaufman;IBM CORPORATION, Intellectual Property Law Dept., P. O. Box 218, Yorktown Heights, NY, 10598, US

(73) Assignee: International Business Machines Corporation, Armonk, NY (US)

(21) Appl. No.: 11/083,729

(22) Filed: Mar. 18, 2005

(51) Int. Cl. A61B 5/02

(52) U.S. Cl. 600/508

(57) ABSTRACT

A method comprising the steps of mounting pressure sensors in a heart-enclosing device; transmitting the data produced by said sensors during actual operation of said heart-enclosing device worn by a specific individual; receiving said sensor signals for subsequent analysis by a computer; creating a stress map based on said sensor-based data; and creating a virtual heart pacer (model) for optimal support and comfort based on the stress map.

### CROSS REFERENCE TO RELATED APPLICATIONS

The instant application is related to U.S. application Ser. No. ______ , filed ______ , 2005 by Levanoni, et al.; and the U.S. application Ser. No. ______ filed ______ , 2005 by Levanoni, et al. These applications are co-pending, commonly assigned, and incorporated by reference herein.

### BACKGROUND OF THE INVENTION

1. Field of the Invention

This invention relates to methodology for utilizing continual sensor-based data to design and adjust heart pacers to fit an individual, in a given dynamic environment, in an optimal manner.

2. Introduction to the Invention

Static fitting techniques to design and construct heart pacers for specific people are known. A plaster cast is taken and the heart pacer is produced based on that plastic impression.

### SUMMARY OF THE INVENTION

In this context, we have discerned that no attention is given to the dynamic workings of the heart in the changing real environment. Specifically, the stresses experienced by the heart during normal operation are not taken into account, nor is the optimum balance, between support and comfort, taken into account.

We have now discovered novel methodology for exploiting the advantages inherent generally in sensing the dynamic workings (stresses) on specific hearts in actual motion, and using the sensor-based data to optimize the design and construction of the desired heart pacers.

Our work proceeds in the following way.

We have recognized that a typical and important paradigm for presently effecting heart pacers construction, is a largely static and subjective, human paradigm, and therefore exposed to all the vagaries and deficiencies otherwise attendant on static and human procedures. Instead, the novel paradigm we have in mind works in the following way:

First, a patient wears a set of pressure and sensors mounted, say, inside a heart-encasing device (harness). These sensors record their associated stesses produced in normal individual motion in its dynamic environment for a prescribed period of time sufficient to capture all possible stress and strain patterns.

The dynamically acquired data are fed into a computer which creates a map of the forces and stresses experienced by the examined heart. This information is used to design an optimal heart pacer which maximizes support and minimizes discomfort, and results in a computer production of virtual heart pacers that offers optimal performance to the examined heart in its normal operation.

A physical heart pacer may then be produced from a model provided by the virtual heart pacer. This physical heart pacer provides maximum support and maximal comfort to its wearer, following the optimal design of the heart pacer.

We now disclose a novel method which can preserve the advantages inherent in the static approach, while minimizing the incompleteness and attendant static nature and subjectivities that otherwise inure in a technique heretofore used.

To this end, in a first aspect of the present invention, we disclose a novel method comprising the steps of:
i) mounting pressure sensors in a heart-enclosing device;
ii) transmitting data produced by said pressure sensors during actual operation of said heart-enclosing device worn by a specific individual;
iii) receiving said sensor signals for subsequent analysis by a computer;
iv) creating a stress-map based on said sensor-based data; and
v) creating a virtual heart pacer (model) for optimal support and comfort based on step v stress-map.

The novel method preferably comprises a further step of actual construction of said physical heart pacer.

### BRIEF DESCRIPTION OF THE DRAWING

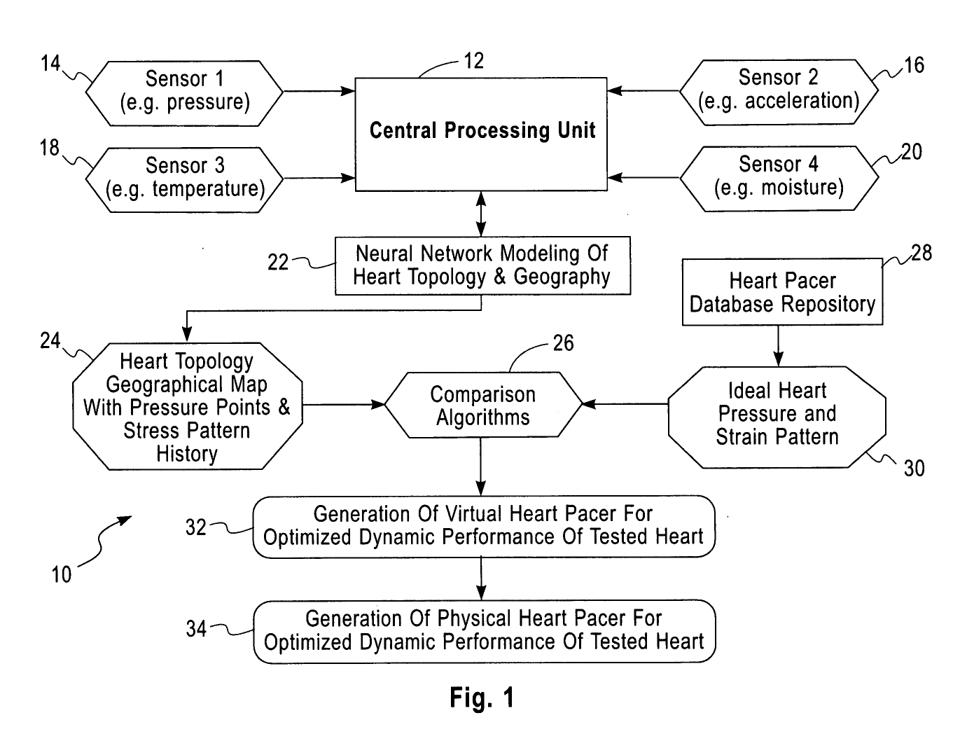

The invention is illustrated in the accompanying drawing, in which FIG. 1 (numerals 10-34) provides an illustrative flowchart comprehending overall realization of the method of the present invention.

### DETAILED DESCRIPTION OF THE PRESENT INVENTION

Typical Application

Attention is now directed to FIG. 1, numerals 10-34.

In a typical case, the patient's heart is fitted with a temporary harness containing a number of sensors, located at prescribed locations on the tested heart. These sensors, which preferably include pressure, temperature and humidity, are connected to a recording device.

The patient is asked to wear the harness for several days and follow his/her normal routine.

During the test period, sensor data are recorded (including time stamps) in the recording device. The patient returns the harness and the recording device at the end of the test period. The information stored in the recording device is then downloaded to a computer which stores all data in a database.

The data are then analyzed by a program (prefearably a neural network modeling program) which creates maps of the tested heart at different times. These maps also contains the sensors' reading at these times. Thus, the system now has information on the dynamic behavior of the tested heart, including parametric information.

Based on these maps and maps of an ideal heart under similar conditions, an optimization program designs an optimized virtual heart pacer for the patient. This design is then fed to a machine which generates an optimized physical heart pacer.

What is claimed:

1. A method comprising the steps of:
i) mounting pressure sensors in a heart-enclosing device;
ii) transmitting the data produced by said sensors during actual operation of said heart-enclosing device worn by a specific individual;
iii) receiving said sensor signals for subsequent analysis by a computer;
iv) creating a stress map based on said sensor-based data; and
v) creating a virtual heart pacer (model) for optimal support and comfort based on step iv stress map.

2. A method according to claim 1, comprising a step of using temperature, moisture, skin conductivity and other sensors which may be correlated with support and comfort of worn heart pacer.

3. A method according to claim 1, comprising a step of using interpolation techniques to completely map stresses experienced by a heart over a period of time.

4. A method according to claim 3, comprising a step of updating the virual heart pacer model using the interpolating map.

5. A method according to claim 3, comprising a step of using the interpolated map to directly design the virtual heart pacer in an optimal manner.

6. A method according to claim 1, comprising a step of using linear or non-linear techniques to model an optimal heart pacer.

7. A method according to claim 6, comprising a step of employing neural networks as the modeling technique.

8. A method according to claim 1, comprising a step of employing regression as the modeling technique.
